(19) United States
(12) Patent Application Publication
(10) Pub. No.: US 2011/0306783 A1
(43) Pub. Date: Dec. 15, 2011

(54) COMPOSITION STABILIZED FOR PURIFICATION AND METHOD FOR PURIFYING AND FOR PRODUCING HYDROXYALKYL (METH)ACRYLATES

(75) Inventors: Dirk Broell, Langen (DE); Benedikt Laux, Monzernheim (DE); Christian Maul, Neustadt a.d. W (DE); Thorben Schuetz, Seeheim-Jugenheim (DE); Bardo Schmitt, Mainz-Kastel (DE); Volker Schleep, Einhausen (DE); Claus Zimmer, Worms (DE)

(73) Assignee: Evonik Roehm GmbH, Darmstadt (DE)

(21) Appl. No.: 13/202,248

(22) PCT Filed: Feb. 25, 2010
(86) PCT No.: PCT/EP10/52362

§ 371 Date: Aug. 18, 2011

(30) Foreign Application Priority Data
Mar. 16, 2009 (DE) 10 2009 001 577.9

(51) Int. Cl. C07C 67/62; C07C 69/54

(52) U.S. Cl. 560/2; 560/218

(57) ABSTRACT

The present invention relates to a composition which comprises at least one hydroxyalkyl(meth)acrylate and has been stabilized for a purification, which comprises hydroquinone monomethyl ether and 4-hydroxy-2,2,6,6-tetramethylpiperidine N-oxyl. The present invention additionally describes processes for purifying and for preparing hydroxyalkyl(meth)acrylates. A further aspect of the present invention is a composition which is obtainable by the processes mentioned.

The present invention relates to a composition stabilized for a purification. The present invention further describes processes for purifying and for preparing hydroxyalkyl(meth)acrylates.

Monomers, for example styrene or (meth)acrylates, tend to undesirably and prematurely polymerize in the course of storage. To prevent this, polymerization inhibitors are therefore typically added to these monomers. A process for stabilizing (meth)acrylates is, for example, the subject of European patent application EP-A-0 620 206. According to this publication, it is possible to stabilize especially (meth)acrylates by means of a mixture of polymerization inhibitors which comprises at least one N-oxyl compound, at least one phenol compound and at least one phenothiazine compound.

Owing to the polymerization tendency detailed above, the reaction mixtures obtained in the preparation of hydroxyalkyl(meth)acrylates are admixed with polymerization inhibitors for purification, though it is also possible to add the polymerization inhibitors during the preparation. One preferred process for purifying hydroxyalkyl(meth)acrylates is described in European patent application EP-A-1 090 904. According to this, a reaction mixture which comprises hydroxyalkyl(meth)acrylates can be purified particularly efficiently by a distillation combined with a thin-film evaporator. The processes detailed in EP-A-1 090 904 lead to relatively pure products in high yields. When, however, the polymerization inhibitors detailed in this publication are used, undesired polymer formation occurs in many cases owing to inadequate stabilization, which can be avoided when large amounts of polymerization inhibitors are used. However, these high amounts are uneconomic, and discoloration of the composition obtained after the purification can occur. For example, when the inhibitor composition described in EP-A-0 620 206 is used, polymerization can reliably be prevented. In the course of storage of the composition obtained, however, undesired discoloration of the product occurs, which necessitates a further purification after a short time.

In view of the prior art, it was thus an object of the present invention to provide a composition which comprises at least one hydroxyalkyl(meth)acrylate and has been stabilized for a purification, from which, after an efficient purification, a hydroxyalkyl(meth)acrylate composition is obtainable, which can be stored in a particularly simple manner without occurrence of significant discoloration.

It was a further object of the present invention to provide processes for preparing and purifying hydroxyalkyl(meth)acrylates, which can be performed simply and reliably. At the same time, the product should as far as possible be obtained in high yields and, viewed overall, with low energy consumption.

It was a further object of the invention to provide a process in which a hydroxyalkyl(meth)acrylate can be obtained very selectively. In addition, the process should provide a very substantially constant product quality.

In addition, a stabilized hydroxyalkyl(meth)acrylate composition should be provided, which exhibits essentially no discoloration after long storage.

These objects, and further objects which are not stated explicitly but can be derived or inferred directly from the connections discussed herein by way of introduction, are achieved by a composition having all features of Claim 1. Appropriate modifications of the inventive composition are protected in the dependent claims which refer back to Claim 1. With regard to a process for purifying or for preparing hydroxyalkyl(meth)acrylates and to a stabilized hydroxyalkyl(meth)acrylate composition, Claims 10, 16 and 18 provide a solution to the underlying problems.

The present invention accordingly provides a composition which comprises at least one hydroxyalkyl(meth)acrylate and has been stabilized for a purification, which is characterized in that the composition comprises hydroquinone monomethyl ether and 4-hydroxy-2,2,6,6-tetramethylpiperidine N-oxyl.

It is thus possible in an unforeseeable manner to provide a composition which comprises at least one hydroxyalkyl(meth)acrylate and has been stabilized for a purification, from which, after an efficient purification, a hydroxyalkyl(meth)acrylate composition is obtainable, which can be stored in a particularly simple manner without occurrence of significant discoloration.

In addition, processes for purifying and preparing hydroxyalkyl(meth)acrylates according to the present invention can be performed simply and reliably, the product being obtainable in high yields and, viewed overall, with low energy consumption.

The processes according to the invention can very selectively provide hydroxyalkyl(meth)acrylates. In addition, the processes provide a very constant product quality.

In addition, a stabilized hydroxyalkyl(meth)acrylate composition obtainable by the process according to the invention exhibits essentially no discoloration after long storage.

According to the invention, a composition which comprises at least one hydroxyalkyl(meth)acrylate and has been stabilized for a purification is provided, the expression “a composition stabilized for a purification” meaning that the composition can be subjected to a purification without occurrence of excessive polymerization.

In a particular aspect of the present invention, a composition stabilized for a purification exhibits a change in the colour number of not more than 30, more preferably not more than 20, after storage at 100° C. for 5 hours. The colour number can be determined especially by the process detailed in DE-A-10 131 479 (determination of the colour by the platinum-cobalt scale; also referred to as APHA or turbidity number), the process for determining the platinum-cobalt colour number detailed in the publication DE-A-10 131 479, filed at the German Patent and Trade Mark Office on 29 Jun. 2001 with application number DE 101 31 479.5, being incorporated into this application for the purposes of disclosure. This process was developed on the basis of DIN EN ISO 6271.

An inventive composition comprises hydroxyalkyl(meth)acrylate. The expression “hydroxyalkyl(meth)acrylates” comprises hydroxyalkyl methacrylates, hydroxyalkyl acrylates and mixtures thereof. Hydroxyalkyl(meth)acrylates are esters of (meth)acrylic acid which are widely known in the technical field, the alcohol radical of which has at least one hydroxyl group. The preferred hydroxyalkyl(meth)acrylates include, for example, 2-hydroxyethyl methacrylate, 2-hydroxyethyl acrylate, hydroxypropyl methacrylate, especially 2-hydroxypropyl methacrylate and 3-hydroxypropyl methacrylate, and/or hydroxypropyl acrylate, especially 2-hydroxypropyl acrylate and 3-hydroxy-propyl acrylate.

The proportion of hydroxyalkyl(meth)acrylate in an inventive composition stabilized for a purification is preferably at least 75% by weight, more preferably at least 95% by weight. This proportion can be determined especially by gas chromatography.

An inventive composition further comprises hydroquinone monomethyl ether (CAS number 150-76-5) and 4-hydroxy-2,2,6,6-tetramethylpiperidine N-oxyl (CAS number 2226-96-2).

The weight ratio of hydroquinone monomethyl ether to 4-hydroxy-2,2,6,6-tetramethylpiperidine N-oxyl is not critical per se. Surprising advantages can be achieved especially by virtue of this weight ratio being in the range from 40:1 to 1:10, preferably in the range from 20:1 to 1:2 and most preferably in the range from 10:1 to 3:1.

Even relatively small amounts of hydroquinone monomethyl ether are sufficient for stabilization, the proportion of hydroquinone monomethyl ether in the composition stabilized for purification being preferably 25 to 1000 ppm, more preferably 35 to 500 ppm. In a particular aspect, it is especially possible to obtain highly stabilized compositions which can be stored over a long period. These compositions preferably feature a proportion of hydroquinone monomethyl ether in the range from 25 ppm to 1000 ppm, especially 50 to 500 ppm, more preferably 100 to 400 ppm and most preferably 150 to 350 ppm. Particular advantages can additionally also be obtained by compositions with lower stabilization, which preferably have a proportion of hydroquinone monomethyl ether in the range from 35 ppm to 100 ppm, more preferably 40 to 80 ppm.

In addition to hydroquinone monomethyl ether, the composition is stabilized using 4-hydroxy-2,2,6,6-tetramethylpiperidine N-oxyl. In a particular modification of the present invention, the proportion of 4-hydroxy-2,2,6,6-tetramethylpiperidine N-oxyl in the composition stabilized for purification may preferably be 20 to 200 ppm, more preferably 30 to 100 ppm and most preferably 40 to 60 ppm.

Surprising improvements can be achieved especially by a composition which has a minimum proportion of N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine, N-1,4-dimethylpentyl-N′-phenyl-p-phenylenediamine, phenothiazine, Nigrosine Base BA and/or 1,4-benzoquinone. It is thus surprisingly possible to prevent discoloration attributable to storage in a purified hydroxyalkyl(meth)acrylate composition. Particular improvements are exhibited especially by compositions which comprise not more than 10 ppm, more preferably not more than 1 ppm and most preferably comprise no measurable proportion of N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine, N-1,4-dimethylpentyl-N′-phenyl-p-phenylenediamine, phenothiazine, Nigrosine Base BA and/or 1,4-benzoquinone.

An inventive composition which has been stabilized for a purification may comprise further polymerization inhibitors. The polymerization inhibitors suitable for this purpose include, for example, tocopherol, preferably α-tocopherol, N,N-diethylhydroxylamine, ammonium N-nitrosophenylhydroxylamine (cupferron) and/or hydroquinone. Particular improvements can be achieved especially by a proportion of these polymerization inhibitors in the range from 10 ppm to 80 ppm, more preferably in the range from 20 ppm to 40 ppm. Compositions of interest are especially those which feature a weight ratio of tocopherol to 4-hydroxy-2,2,6,6-tetramethylpiperidine N-oxyl in the range of preferably 10:1 to 1:10, more preferably in the range from 2:1 to 1:4. The weight ratio of N,N-diethylhydroxylamine to 4-hydroxy-2,2,6,6-tetramethylpiperidine N-oxyl is preferably within the range from 10:1 to 1:10, more preferably in the range from 2:1 to 1:4. In a further aspect of the present invention, the weight ratio of ammonium N-nitrosophenylhydroxylamine (cupferron) to 4-hydroxy-2,2,6,6-tetramethylpiperidine N-oxyl may be in the range from 10:1 to 1:10, more preferably in the range from 2:1 to 1:4. Further preferred compositions of interest are those in which the weight ratio of hydroquinone to 4-hydroxy-2,2,6,6-tetramethylpiperidine N-oxyl is preferably in the range from 10:1 to 1:10, more preferably in the range from 2:1 to 1:4.

An inventive composition can be used especially in a process for purifying hydroxyalkyl(meth)acrylates, which likewise forms part of the subject-matter of the present invention.

To improve the inhibiting action of the polymerization inhibitors present in the composition, oxygen can additionally be used. It can be used, for example, in the form of air, the amounts metered in advantageously being such that the content in the gas phase above the reaction mixture remains below the explosion limit. Preference is given here to amounts of air in the range from 0.1 to 10, more preferably 1 to 5 and most preferably 2 to 4 standard litres per hour and kg of the composition stabilized for a purification. It is equally possible to use inert gas-oxygen mixtures, for example nitrogen-oxygen or argon-oxygen mixtures.

In a particular configuration, a composition stabilized for purification can be treated with oxygen before the purification which can be effected, for example, by distillation. To this end, especially air can be passed through the composition to be purified. By virtue of this configuration, gaseous or volatile constituents can be removed from the composition before it is subjected to a further purification, preferably a distillation.

For the purification, preference is given to using a plant which comprises a still. In this case, surprising advantages can be achieved especially by a column with a low separating performance. This configuration allows especially the yield and the energy efficiency of the plant to be improved. Accordingly, the column used has at most 4, more preferably at most 3, plates. In a particular aspect, a column with preferably at least 2 plates is used.

The number of plates in the present invention refers to the number of trays in a tray column or the number of theoretical plates in the case of a column with structured packing or a column with random packing.

In a particular configuration of the present invention, it is possible to use a column which has separating internals and corresponds to about one plate.

The composition of the present invention, which has been stabilized for a purification, can be fed in above or below the internals detailed above, and it is also possible, according to the type thereof, to introduce the inventive composition within the region of the internals. One way of achieving particular advantages is to feed the composition of the present invention into the column above the internals. The expression “above the internals” means that the high-boiling constituents of the composition introduced are conducted through the internals before they are withdrawn from the column. This can especially achieve advantages with regard to the yields and the purity of the purified composition. In addition, the process can be performed particularly efficiently.

The column of the present invention can be operated with or without a column return stream, a particularly high purity surprisingly being achievable through an embodiment without a column return stream. These advantages can preferably be achieved by feeding the composition of the present invention into the column above any internals present.

In a particular aspect of the present invention, the column is preferably operated at a gas loading factor of at most 2 Pa^{0.5}. Appropriately, the gas loading factor at which the second evaporator is operated is preferably in the range from 0.8 to 1.8 Pa^{0.5}. The gas loading factor (F factor) is calculated from the gas velocity based on the empty cross section of the pipe for withdrawing the gas multiplied by the root of the gas density.

The distillation is performed preferably at a temperature in the range from 40 to 130° C., more preferably in the range from 60 to 110° C. and most preferably 80 to 95° C., these figures being based on the bottom temperature. The pressure at which the distillation is effected may preferably be in the range from 0.1 to 20 mbar absolute, more preferably in the range from 0.5 to 10 mbar and most preferably 1 to 5 mbar absolute, these figures being based on the column top pressure.

To enhance the yield, a portion of the composition obtained from the bottom of the still can be converted to the gas phase with at least one evaporator, for example a thin-film evaporator or a circulation evaporator, and fed into the still. Accordingly, a preferred plant for performing the process for purifying hydroxyalkyl(meth)acrylates has a thin-film evaporator and/or a circulation evaporator.

A particularly preferred plant for purification is described, more particularly, in publication EP-A-1 090 904, filed on 5 Oct. 2000 at the European Patent Office with application number 00121755.3, reference being made to this publication for disclosure purposes and the plants disclosed therein for purification of hydroxyalkyl(meth)acrylates being incorporated into this application.

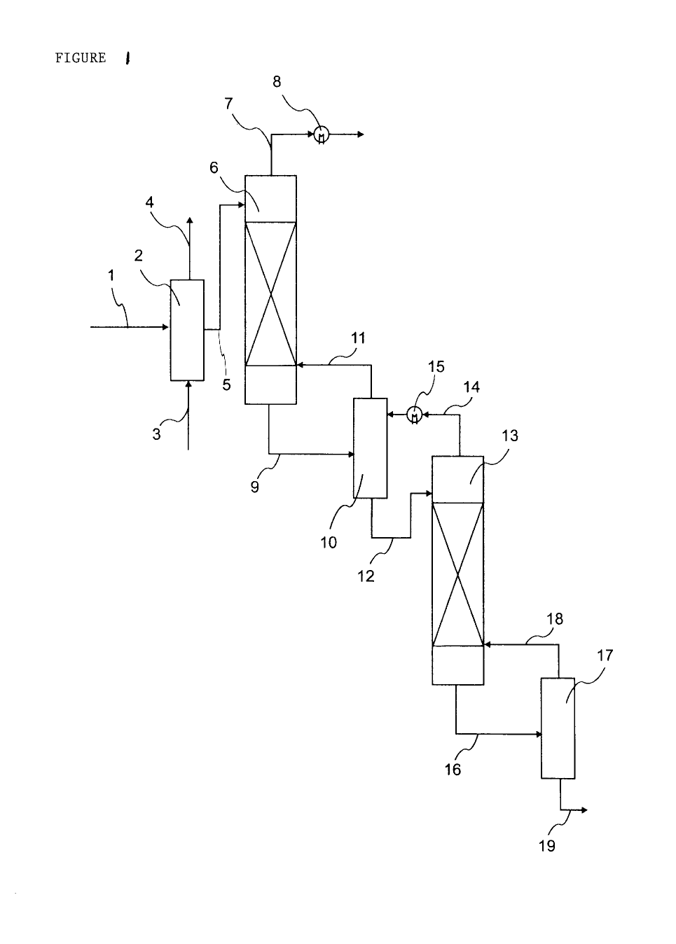

A particularly preferred plant for purifying hydroxyalkyl(meth)acrylates is additionally explained in detail with reference to the appended FIG. 1.

FIG. 1 is a schematic diagram of a plant suitable for purifying hydroxyalkyl(meth)acrylates, without any intention that this should impose a restriction. The plant shown in FIG. 1 can be supplied with a composition comprising hydroxyalkyl(meth)acrylates via an inlet 1. In the present embodiment, inlet 1 leads into a vessel 2 in which the composition to be purified is treated with oxygen. To this end, air can be introduced into vessel 2 via inlet 3, which is discharged from the vessel 2 via outlet 4. The composition treated with oxygen is conducted out of vessel 2 via line 5 into the distillation column 6 which is provided in the present case with internals which preferably correspond to about one plate in separation technology terms. The composition is preferably fed in above the internals, as indicated schematically in the drawing.

The top product is withdrawn from the plant via line 7 of the distillation column 6, in which a cooler 8 is provided.

To enhance the yield, the bottoms of the distillation column 6 are introduced via line 9 into a thin-film evaporator 10, the gaseous products obtained in the thin-film evaporator 10 being fed via line 11 to the distillation column 6. The proportion of the composition fed in which has not been evaporated in the thin-film evaporator 10 is fed via line 12 to a second distillation column 13 which in the present case is likewise provided with internals which correspond to about one plate in separation technology terms. The composition is preferably fed in above the internals, as indicated schematically in the drawing.

The top product obtained from the second still 13 is introduced into the thin-film evaporator 10 via line 14 in which a cooler 15 is provided. The bottoms of the second still 13 are fed via line 16 to a second thin-film evaporator 17. The gaseous products obtained are introduced via line 18 into the second distillation column 13. The by-products obtained are removed from the plant via line 19.

The composition stabilised for a purification can preferably be obtained by a reaction which serves to prepare hydroxyalkyl(meth)acrylates. Such processes which comprise an inventive purification likewise form part of the subject-matter of the present invention. The composition detailed above can preferably be obtained by reacting (meth)acrylic acid with at least one epoxide in the presence of a catalyst.

In the context of the present invention, the term “(meth)acrylic acid” encompasses especially methacrylic acid, acrylic acid and mixtures thereof. In addition to (meth)acrylic acid, an epoxide is used as a second reactant. The preferred epoxides include especially ethylene oxide and propylene oxide.

The molar ratio of (meth)acrylic acid to epoxide may, for example, be within the range from 2:1 to 1:2, more preferably in the range from 0.9:1 to 1:1.1.

Catalysts are preferably used for the reaction. Preferred catalysts are detailed, inter alia, in EP-A-1 231 204, filed on 31 Jan. 2002 at the European Patent Office with the application number EP 02002363.6, the disclosure of this document, especially the catalysts and processes for preparing hydroxyalkyl(meth)acrylates described therein, being incorporated into the present application for the purposes of disclosure.

The conversion, based on (meth)acrylic acid, is preferably at least 95 mol %, more preferably at least 99 mol % and most preferably at least 99.5 mol %. The conversion can be adjusted especially via the reaction time and the reaction temperature.

The reaction preferably takes place at a temperature in the range from 50 to 100° C., more preferably in the range from 60 to 80° C. The preparation can be effected continuously or batchwise. The reaction time of batchwise processes is preferably in the range from 2 to 10 hours, more preferably 4 to 8 hours. The residence time in continuous processes may preferably be within the range from 1 minute to 60 minutes, more preferably in the range from 2 minutes to 30 minutes. The pressure used to prepare the hydroxyalkyl(meth)acrylate is preferably in the range from 0.5 to 25 bar, more preferably in the range from 1 to 3 bar.

The reaction of (meth)acrylic acid with epoxide can be effected either continuously or batchwise. The process for preparing hydroxyalkyl(meth)acrylates can be performed in bulk, i.e. without use of a further solvent. If desired, an inert solvent can also be used.

The amounts and weight ratios of polymerization inhibitors detailed above, especially of hydroquinone monomethyl ether and 4-hydroxy-2,2,6,6-tetramethylpiperidine N-oxyl, can be added already before or during the reaction of (meth)acrylic acid with an epoxide. Owing to the degradation of the polymerization inhibitors during the reaction, however, the amounts added may be higher if anything, in which case the proportion of hydroquinone monomethyl ether added to the reaction mixture for the reaction of (meth)acrylic acid with epoxides is preferably in the range from 25 to 1000 ppm, more preferably 100 to 500 ppm. Accordingly, it is also possible to add slightly greater amounts of 4-hydroxy-2,2,6,6-tetramethylpiperidine N-oxyl in this reaction, in which case the proportion of 4-hydroxy-2,2,6,6-tetramethylpiperidine N-oxyl added to the reaction mixture for the preparation of hydroxyalkyl(meth)acrylate from (meth)acrylic acid and epoxides may preferably be within the range from 20 to 200 ppm, more preferably in the range from 30 to 80 ppm.

The reaction mixture for preparing hydroxyalkyl(meth)acrylate preferably comprises not more than 10 ppm, more preferably not more than 5 ppm and most preferably not more than 1 ppm of N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine, N-1,4-dimethylpentyl-N′ phenyl-p-phenylenediamine, phenothiazine, Nigrosine Base BA and/or 1,4-benzoquinone. This can surprisingly prevent a discoloration, attributable to storage, of a purified hydroxyalkyl(meth)acrylate composition.

In a further modification of the present invention, the polymerization inhibitors present in the composition stabilized for a purification can be added after the reaction but before the purification.

The hydroxyalkyl(meth)acrylate composition obtainable by the process exhibits properties unknown to date, and so likewise forms part of the subject-matter of the present invention.

One of these properties is especially a long storability, which can be achieved with no change in the substance properties. For example, the colour number of the composition after storage for at least 180 days at 30° C. is not more than 20, more preferably not more than 10, measured by the process detailed in DE-A-10 131 479.

The proportion of hydroxyalkyl(meth)acrylate in an inventive composition obtainable by the process detailed above is preferably at least 97% by weight, more preferably at least 98% by weight. This proportion can be determined especially by gas chromatography.

The hydroxyalkyl(meth)acrylate composition after the purification detailed above preferably comprises 20 to 80 ppm, more preferably 30 to 50 ppm, of hydroquinone monomethyl ether and 0.1 to 2 ppm, more preferably 1 to 2 ppm, of 4-hydroxy-2,2,6,6-tetramethylpiperidine N-oxyl.

The weight ratio of hydroquinone monomethyl ether to 4-hydroxy-2,2,6,6-tetramethylpiperidine N-oxyl in a hydroxyalkyl(meth)acrylate composition obtainable in accordance with the invention is preferably in the range from 100:1 to 10:1, more preferably in the range from 40:1 to 20:1.

In a particular configuration, the hydroxyalkyl(meth)acrylate composition after the purification detailed above may comprise 1 to 50 ppm, more preferably 5 to 25 ppm, of tocopherol, preferably α-tocopherol.

The tocopherol compounds usable in the context of the invention are chroman-6-ols substituted in the 2 position by a 4,8,12-trimethyltridecyl radical (3,4-dihydro-2H-1-benzopyran-6-ols). The tocopherols usable with preference in accordance with the invention include alpha-tocopherol, beta-tocopherol, gamma-tocopherol, delta-tocopherol, zeta2-tocopherol and eta-tocopherol, all of the aforementioned compounds each in the (2R,4′R,8′R) form, and alpha-tocopherol in the (all-rac) form. Preference is given to alpha-tocopherol in the (2R,4′R,8′R) form (trivial name: RRR-alpha-tocopherol) and the synthetic racemic alpha-tocopherol (all-rac-alpha-tocopherol). Among these, the latter is in turn of particular interest owing to the relatively low cost.

A hydroxyalkyl(meth)acrylate composition obtainable by an inventive purification preferably does not comprise any N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine, N-1,4-dimethylpentyl-N′-phenyl-p-phenylenediamine, phenothiazine, Nigrosine Base BA and/or 1,4-benzoquinone.

A hydroxyalkyl(meth)acrylate composition which has been obtained according to the present invention can preferably be used to prepare polymers. These compositions exhibit, with equal stabilization, especially an equal proportion of hydroquinone monomethyl ether, an improvement in the colour number, which can be determined by the process detailed in DE-A-10 131 479, after a customary polymerization which can preferably be effected at temperatures less than or equal to 180° C., preferably less than or equal to 160° C. and most preferably less than or equal to 140° C. If a colour number which is also achieved with prior art compositions can be tolerated, compositions obtainable in accordance with the invention require lower stabilization for this purpose, more particularly a lower proportion of hydroquinone monomethyl ether. Lower stabilization allows further advantages to be achieved.

The present invention will be illustrated hereinafter with reference to examples and comparative examples, without any intention that this should impose a restriction.

### EXAMPLE 1

A composition comprising 2-hydroxyethyl methacrylate (HEMA) was prepared according to the present invention, using, for preparation and purification, a stabilizer mixture which comprises 200 ppm of hydroquinone monomethyl ether and 50 ppm of 4-hydroxy-2,2,6,6-tetramethylpiperidine N-oxyl. The purification was effected in a plant illustrated in detail in FIG. 1, which comprised two distillation columns and two thin-film evaporators.

After the purification, a composition which contained approx. 50 ppm of hydroquinone monomethyl ether and approx. 1 ppm of hydroxy-2,2,6,6-tetramethylpiperidine N-oxyl was obtained. The colour number of the composition prepared was less than 5.

The storability of this composition was measured via the determination of the colour number, by performing different tests. The colour number was measured by the process detailed in DE-A-10 131 479. Thus, the storability was measured at 30° C. over 6 months. To this end, 25 g of the composition were transferred into a 30 ml bottle (brown, wide-necked). It was stored in a forced-air drying cabinet at 30° C. The colour number was measured after 6 months.

In addition, in a short test, the colour number was determined after storage at 100° C. over 5 hours.

As the application test, the colour number of a standard clearcoat (solvent-based) was measured. This was prepared by polymerizing a monomer mixture at temperatures of >140° C. under a nitrogen atmosphere within 4-6 h. The solids content of the formulation was approx. 62%, the proportion of HEMA in the copolymer about 30%.

The data obtained are shown in Table 1.

### EXAMPLE 2

Example 1 was essentially repeated, except that the proportion of hydroquinone monomethyl ether in the composition obtained after the purification was increased from 50 ppm to 200 ppm. The colour number of the composition prepared was less than 5.

The above-described tests were performed, and the data obtained are shown in Table 1.

### EXAMPLE 3

Example 1 was essentially repeated, except that 20 ppm of tocopherol were additionally added to the composition obtained after the purification. The colour number of the composition prepared was less than 5.

Some of the above-described tests were carried out, and the data obtained are shown in Table 1.

TABLE 1

| | Colour number after | Colour number after | Colour number |
|---|---|---|---|
| | 6 months at 30° C. | 5 hours at 100° C. | in a clearcoat |
| Example 1 | 6 | 16 | 20 |
| Example 2 | <5 | 11 | 8 |
| Example 3 | <5 | <5 | |

1. A composition, comprising:
at least one hydroxyalkyl(meth)acrylate and has been stabilized for a purification;
hydroquinone monomethyl ether; and
4-hydroxy-2,2,6,6-tetramethylpiperidine N-oxyl.

2. The composition of claim 1, wherein the hydroxyalkyl(meth)acrylate comprises at least one selected from the group consisting of 2-hydroxyethyl methacrylate, 2-hydroxyethyl acrylate, hydroxypropyl methacrylate, and hydroxypropyl acrylate.

3. The composition of claim 1, wherein a weight ratio of hydroquinone monomethyl ether to 4-hydroxy-2,2,6,6-tetramethyl-piperidine N-oxyl is within a range from 20:1 to 1:1.

4. The composition of claim 1, wherein a proportion of hydroquinone monomethyl ether in the composition is 25 to 1000 ppm.

5. The composition of claim 1, wherein a proportion of 4-hydroxy-2,2,6,6-tetramethyl-piperidine N-oxyl in the composition is 20 to 200 ppm.

6. The composition of claim 1, comprising not more than 10 ppm of at least one selected from the group consisting of N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine, N-1,4-dimethylpentyl-N′-phenyl-p-phenylenediamine, phenothiazine, Nigrosine Base BA, and 1,4-benzoquinone.

7. The composition of claim 1, comprising at least one selected from the group consisting of tocopherol, N, N-diethylhydroxylamine, ammonium N-nitrosophenylhydroxylamine (cupferron), and hydroquinone.

8. The composition of claim 7, wherein a proportion of the at least one selected from the group consisting of tocopherol, N,N-diethylhydroxylamine, ammonium N-nitrosophenylhydroxylamine (cupferron), and hydroquinone, in the composition is in a range from 10 to 80 ppm.

9. The composition of claim 7, wherein a weight ratio of the at least at one selected from the group consisting of tocopherol, N,N-diethylhydroxylamine, ammonium N-nitrosophenylhydroxylamine (cupferron), and hydroquinone, to 4-hydroxy-2,2,6,6-tetramethylpiperidine N-oxyl is in a range from 10:1 to 1:10.

10. A process for purifying at least one hydroxyalkyl(meth)acrylate, the method comprising purifying the composition of claim 1.

11. The process of claim 10, wherein the composition is purified in a plant which comprises a still.

12. The process of claim 11, wherein a column of the still comprises at most 4 plates.

13. The process of claim 11, wherein the purifying is effected by performing a distillation with a temperature in a range from 60 to 110° C.

14. The process of claim 11, wherein the column of the still has internals and the composition is fed into the column of the still above the internals.

15. The process of claim 10, wherein the composition is purified in a plant which comprises at least one selected from the group consisting of a thin-film evaporator and a circulation evaporator.

16. A process for preparing at least one hydroxyalkyl(meth)acrylate, the method comprising:
reacting (meth)acrylic acid with at least one epoxide in the presence of a catalyst, to obtain a resulting mixture; and
purifying the resulting reaction mixture by performing the process of claim 10.

17. The process of claim 16, the reaction mixture comprises not more than 10 ppm of at least one selected from the group consisting of N,N′-diphenyl-p-phenylenediamine, N,N′-di-2-naphthyl-p-phenylenediamine, N,N′-di-p-tolyl-p-phenylene-diamine, N-1,3-dimethylbutyl-N′-phenyl-p-phenylenediamine, N-1,4-dimethylpentyl-N′-phenyl-p-phenylenediamine, phenothiazine, Nigrosine Base BA, and 1,4-benzoquinone.

18. A stabilized hydroxyalkyl(meth)acrylate composition, obtained by the process of claim 10.

19. The composition of claim 18, comprising 30 to 50 ppm of hydroquinone monomethyl ether and 0.1 to 2 ppm of 4-hydroxy-2,2,6,6-tetramethylpiperidine N-oxyl.

20. The composition of claim 18, having a color number after storage for at least 180 days at 30° of not more than 20.

21. The composition of claim 17, comprising tocopherol.
